(19) United States
(12) Patent Application Publication
(10) Pub. No.: US 2023/0330180 A1
(43) Pub. Date: Oct. 19, 2023

(54) ANTIVIRAL COMPOSITIONS AND METHODS

(71) Applicant: Bexion Pharmaceuticals Inc., Covington, KY (US)

(72) Inventors: Gilles Hughes Tapolsky, Boulder, CO (US); Ray Takigiku, Covington, OH (US); Nilabh Chaudhary, West Chester, OH (US)

(73) Assignee: Bexion Pharmaceuticals Inc, Covington, KY (US)

(21) Appl. No.: 18/151,910

(22) Filed: Jan. 9, 2023

Related U.S. Application Data
(60) Provisional application No. 63/297,820, filed on Jan. 10, 2022.

(51) Int. Cl. A61K 38/17; A61K 31/675; A61K 47/24; A61P 31/14

(52) U.S. Cl. CPC A61K 38/1709; A61K 31/675; A61K 47/24; A61P 31/14

(57) ABSTRACT

Disclosed are compositions containing a saposin C polypeptide and a phospholipid and methods that are useful for treating viral infections.

### CROSS REFERENCE TO RELATED APPLICATION

This application claims the benefit of U.S. Provisional Application No. 63/297,820, filed on Jan. 10, 2022.

### REFERENCE TO AN ELECTRONIC SEQUENCE LISTING

The contents of the electronic sequence listing (19906003US1 SEQ LIST ST26.xml; Size 2 KB; and Date of Creation Jan. 9, 2023) is herein incorporated by reference in its entirety.

### FIELD OF THE INVENTION

The present invention relates to antiviral compositions for the prevention or reducing the incidence of and/or treatment of various viral conditions.

### BACKGROUND OF THE INVENTION

Many fatal infectious diseases that afflict mankind are caused by viruses. Viruses can be DNA-based or RNA-based. Examples of disease-causing viruses include: chikungunya (alphavirus); other viruses of the alphavirus (e.g., Venezuelan equine encephalitis virus); marburg (Marburgvirus); dengue (flavivirus); Epstein-Barr virus (Lymphocryptovirus); Hepatitis A (Hepatovirus); Hepatitis B (Orthohepadnavirus); Hepatitis C (Hepacivirus); Hepatitis E (Hepevirus); Hepatitis delta viruses (Deltavirus); herpesviruses 1 and 2 (Simplexvirus); herpesviruses 6 and 7 (Roseolovirus); herpesvirus 8 (Rhadinovirus); Human immunodeficiency virus (Lentivirus); papillomavirus 1 (Mupapillomavirus); papillomaviruses 2, 16, 18 (Alphapapillomavirus); Influenza A virus (Influenzavirus A); Influenza B virus (Influenzavirus B); Influenza C virus (Influenzavirus C); Influenza D virus (Influenzavirus D); Merkel cell polyomavirus (Polyomavirus); measles virus (Morbilivirus); mumps virus (Rubulavirus); rhinovirus (A, B, C), poliovirus, coxsackievirus, echovirus, enterovirus 68, 70, respiratory syncytial virus A and B (Enterovirus); rabies virus (Lyssavirus); rotaviruses A, B, or C (Rotavirus); rubella virus (Rubivirus); seoul virus (Hantavirus); simian foamy virus (Simiispumavirus); Simian virus 5 (Rubulavirus); zika virus or West Nile Virus (Flavivirus); Varicella-zoster virus (i.e., chickenpox; Varicellovirus); Variola virus (i.e., smallpox; Orthopoxvirus); Nipah virus (Henipavirus); coronaviruses (i.e, SARS, MERS, COVID-19; α-coronavirus, β-coronavirus, γ-coronavirus and δ-coronavirus); and many more (see, e.g., https://viralzone.expasy.org/678).

Vaccines are one of the most effective ways of preventing and treating diseases caused by viruses. Unfortunately, many research and clinical vaccine programs have a low probability of success.

Thus, there is a need for new strategies to develop effective treatments for preventing and treating diseases caused by viruses.

### SUMMARY OF THE INVENTION

This disclosure pertains to compositions comprising saposin C and methods of using such compositions in the treatment of infections caused by various viruses.

Provided are methods of treating a viral infection in a subject, the method including identifying a subject having a viral infection and administering to the subject a therapeutically effective amount of an active agent including a saposin C polypeptide and a phospholipid.

In some instances, also provided are methods of reducing the incidence of a viral infection in a subject, the method including administering to the subject a prophylactically effective amount of an active agent including a saposin C polypeptide and a phospholipid.

Also described are methods of treating a viral infection in a subject, the method including administering to the subject a therapeutically effective amount of 1) an active agent including a saposin C polypeptide and a phospholipid, and 2) an antiviral agent. In some embodiments the antiviral agent is remdesivir.

Also provided are, methods of reducing the incidence of a viral infection in a subject, the method including administering to the subject a prophylactically effective amount of 1) an active agent comprising a saposin C polypeptide and a phospholipid, and 2) an antiviral agent. In some embodiments the antiviral agent is remdesivir.

In some embodiments, in any of the methods described, the saposin C polypeptide includes the amino acid sequence of SEQ ID NO: 1 with zero to four amino acid insertions, substitutions, or deletions.

In some embodiments, in any of the methods described, the saposin C polypeptide comprises the amino acid sequence of SEQ ID NO: 1.

In some embodiments, in any of the methods described, the saposin C polypeptide consists of the amino acid sequence of SEQ ID NO: 1.

In some embodiments, in any of the methods described, the phospholipid has a net negative charge at neutral pH.

In some embodiments, in any of the methods described, the phospholipid is a phosphoglyceride.

In some embodiments, in any of the methods described, the phosphoglyceride is a phosphatidate or phosphatidylserine.

In some embodiments, in any of the methods described, the phosphoglyceride is phosphatidylserine. In some instances, the phosphatidylserine comprises one or more of dioleoyl phosphatidylserine (DOPS), dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, or diphytanoyl phosphatidylserine lipid, or a salt of any of the above. In some embodiments, the phosphatidylserine is DOPS or a salt thereof.

In some embodiments, in any of the methods described, the molar ratio of the saposin C polypeptide to the phospholipid is in the range of 1:2 to 1:50. In some instances, the ratio of the saposin C polypeptide to the phospholipid is 1:12.

In some embodiments, in any of the methods described, the viral infection is an infection by a virus selected from: SARS-CoV, MERS-CoV, SARS-CoV-2, Rhinovirus A, Rhinovirus B, Rhinovirus C, Poliovirus, Coxsackievirus, Echovirus, Enterovirus, Respiratory syncytial virus A, Respiratory syncytial virus B, Influenza A virus, Influenza B virus, Influenza C virus, Influenza D virus, Dhori virus, Ebolavirus, Marburgvirus, Nipah virus, a virus of the Flaviviridae family, and a virus of the Togaviridae family. In some embodiments, the virus is Influenza A virus, Influenza B virus, Influenza C virus, or Influenza D virus. In some instances, the virus is SARS-CoV-2. In some embodiments, the virus is Respiratory syncytial virus A or Respiratory syncytial virus B.

In some embodiments, in any of the methods described, the active agent is administered intravenously in a dose of 2.3-3.2 mg/kg saposin C polypeptide.

In some embodiments, in any of the methods described, a dose of the active agent is administered to the subject each day for at least three consecutive days.

In some embodiments, in any of the methods described, the subject is a human.

Unless otherwise defined, all technical and scientific terms used in the present specification have the same meaning as commonly understood by one of ordinary skill in the art to which this invention belongs. Methods and materials are described for use in the present invention; other suitable methods and materials known in the art can also be used. The materials, methods, and examples are illustrative only and not intended to be limiting. All publications, patent applications, patents, sequences, and other references mentioned in the present specification are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph illustrating an inhibition curve of BXQ-350 in MDCK cells infected with Influenza AH1N1. The antiviral activity of BXQ-350 was calculated based on the protection of the virus-induced CPE at each concentration normalized by the virus control.

FIG. 2 is a graph showing that BXQ-350 is not cytotoxic to normal cells.

FIG. 3 is a graph illustrating the synergism of BXQ-350 and Remdesivir when used to treat cells infected with the HCoV229E strain.

FIG. 4 is a graph illustrating the prophylactic properties of (duplicate) BXQ-350. Triangles are cell viability results showing that BXQ-350 is not cytotoxic to normal cells in these assay conditions. Circles represent the percentage of cells not virally infected.

FIG. 5 is a graph illustrating the prophylactic properties of (duplicate) BXQ-350. Triangles are cell viability results showing that BXQ-350 is not cytotoxic to normal cells in these assay conditions. Circles represent the percentage of cells not virally infected.

FIG. 6 is a graph showing ability of BXQ-350 to inhibit viral replication of the VEEV TC-83 strain.

FIG. 7 is a graph showing the effect of BXQ-350 on the viral load of the VEEV TrD strain.

FIG. 8 is a graph showing the associated percent inhibition against the VEEV TrD strain.

### DETAILED DESCRIPTION

The present invention relates to compositions and methods for treating and preventing viral infections.

### Compositions

### Description of Active Agent

The active agent used in the present methods includes a saposin C (“SapC”) polypeptide and a phospholipid, which together form lipid vesicles (also termed “liposomes” or “nanovesicles”) when suspended in aqueous solution. The SapC polypeptide consists of or comprises the amino acid sequence of SEQ ID NO: 1, or comprises SEQ ID NO: 1 with one to four amino acid alterations (insertions, substitutions, or deletions): for example, an insertion of one to four amino acid residues into SEQ ID NO: 1; or substitution of one, two, three, or four residues in SEQ ID NO: 1; or a deletion of one, two, three, or four residues from the amino terminus, or from the carboxy terminus, or at an internal site of SEQ ID NO: 1.

Phospholipids that can be incorporated into the active agent include phospholipids that have a net negative charge at neutral pH, e.g., phosphoglycerides such as phosphatidate (diacylglycerol 3-phosphate) and phosphatidylserine. The two fatty acids attached to the phosphoglyceride backbone can be the same or different; can have, e.g., zero, one, or two carbon-carbon double bonds in each carbon chain; and can have carbon chains from C6 up to C20, e.g., oleic acid, hexanoic acid, octanoic acid, decanoic acid, lauric acid, myristoic acid, stearic acid, palmitic acid, linoleic acid, and phytanic acid, and combinations of any two of these. Thus, where the phospholipid is phosphatidyl serine, the following are typical examples: dioleoyl phosphatidylserine (DOPS), dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, and diphytanoyl phosphatidylserine lipid. The active agent can include a combination of any two or more (e.g., two or three) different phospholipids, such as two or three different phosphatidylserine lipids. (The terms “phosphatidylserine” and “phosphatidylserine lipid” are used interchangeably.)

In aqueous compositions at neutral pH, phospholipids typically exist in the form of a salt with a cation, and so references to DOPS and other phospholipids used in the present compositions are meant to include both the salt and non-salt forms of the phospholipids. Suitable cations include any pharmaceutically acceptable cation that forms a salt with the phospholipid, such as any of the following: ammonium ion; L-arginine ion; benzathine ion; deanol ion; diethanolamine (2,2′-iminodiethanol) ion; hydrabamine ion; lysine ion; potassium ion; sodium ion; triethanolamine (2,2′,2″-nitrilotri(ethan-1-ol)) ion; and tromethamine (2-amino-2-(hydroxymethyl)propane-1,3-diol) ion. The sodium, potassium, and ammonium salts are typical.

The molar ratio of the SapC polypeptide to the phospholipid in a composition of the invention can be in the range from 1:2 to 1:50, for example 1:5 to 1:30, or 1:8 to 1:20, or 1:11 to 1:13. Suitable molar ratios include but are not limited to 1:10, 1:11, 1:12, 1:13, 1:14, and 1:15. The mass ratio of the polypeptide to the phosphatidlyserine lipid is in the range from about 1:0.11 to 1:4.8, or about 1:0.18 to about 1:4.5, or about 1:0.45 to about 1:2.7, or about 1:0.72 to about 1:1.81, or about 1:1 to about 1:1.2.

The active agent may be supplied in the form of a solid (e.g., a lyophilized powder) with or without pharmaceutically acceptable buffers and other inactive ingredients. The solid is typically reconstituted in sterile water or saline before administration, forming a suspension of liposomes in aqueous solution.

When the active agent is in the form of liposomes suspended in an aqueous solution, that solution can also contain pharmaceutically acceptable buffers and other inactive ingredients. Suitable formulations (and methods for preparing them) include those described in U.S. Pat. No. 10,682,411, which is incorporated by reference in its entirety. One example of such a suspension of the active agent in aqueous solution is designated “BXQ-350”. It contains a saposin C polypeptide consisting of SEQ ID NO: 1 and a phospholipid consisting of a sodium salt of DOPS in a molar ratio (SapC to DOPS) of approximately 1:12 (i.e., in the range of 1:11 to 1:13), and the pH is 7.2 ± 0.4. In a specific instance, the formulation includes: SapC at a concentration of 2.2 mg/mL, DOPS at a concentration of 2.4 mg/mL, Tris at a concentration of 25 mM, and Trehalose dihydrate at a concentration of 5% by weight. BXQ-350 is currently being tested as an anticancer therapeutic. This drug targets phosphatidylserine and de novo biosynthesis of ceramide in tumor cells. BXQ-350 has a high affinity for phosphatidylserine-rich membranes in vitro and in vivo. It can induce apoptosis and/necrosis in target cancer cells,but appears to be safe for non-neoplastic cells.

Administration of the active agent is typically via injection (e.g., infusion) of the suspension, and may be by any suitable injectable route, e.g., intravenous, intra-arterial, ocular, intradermal, intramuscular, intra-cardiac, intracranial, subcutaneous, or intraperitoneal. In appropriate situations, dry particles of the active agent, or a solution containing the active agent, could be aerosolized or nebulized and delivered via inhalation through the nose or mouth, or the solution could be delivered as liquid, e.g. eye drops, nasal drops or spray. Also contemplated is oral delivery, e.g., as a mouthwash or gargle or sublingual formulation, or swallowed as a liquid, capsule, or tablet. Further details regarding routes of administration can be found, for example, in U.S. Pat. No. 7,834,147.

Administration can occur at least once a day for some number of consecutive days, e.g., for 3, 4, 5, 6, 7, 8, 9, or more consecutive days, or can be, e.g., every other day, or 3 times a week, or once every 7 ± 3 days, or once every 14 ± 3 days, or once every 28 ± 3 days. The timing of administrations can start with one of those schedules and after a suitable period of treatment change to another that is more or less frequent. The entire period of treatment may be completed in, e.g., one to three or eight or twelve or sixteen weeks, or up to six months, but may continue as long as the patient appears to be benefiting from the treatment and/or if the patient has been or is suspected to have been exposed to a virus.

### Methods of Treatment or Methods of Preventing a Viral Infection

Provided here are methods of treating a subject suffering from a viral infection by administering to the subject a therapeutically effective amount of compositions including the active agent, as described above.

Also provided here are methods of preventing a viral infection from occurring in a subject by administering to the subject a prophylactically effective amount of compositions including the active agent, as described above.

In some embodiments, compositions including the active agent may be used in combination with one or more of: a standard of care treatment, or a second agent, such as an antiviral agent. Suitable antiviral agents include: viral attachment blockers that prevent attachment to target cells, e.g., neuraminidase inhibitors for influenza viruses, including oseltamivir and zanamivir; viral entry inhibitors that block the penetration of the virus into the cell, e.g., Enfuvirtide; protease inhibitors, e.g., nelfinavir and darunavir; uncoating inhibitors, e.g., rimantadine, that block the viral capsid from unraveling, thus preventing the genetic material from being released inside the cell; viral DNA polymerase inhibitors, which block the replication of the viral genome, e.g., acyclovir, valaciclovir, and valganciclovir, used for herpes simplex virus, chickenpox, and cytomegalovirus; viral RNA polymerase inhibitors, e.g., broad spectrum antivirals ribavirin used for treating RSV, HCV, and some hemorrhagic fevers, and remdesivir used for COVID-19; nucleoside and nucleotide reverse transcriptase inhibitors, e.g., tenofovir and lamivudine, used for treating hepatitis B virus and HIV; non-nucleoside reverse transcriptase inhibitors, e.g., nevirapine, used for treating HIV; integrase inhibitors, which block the pro-viral DNA from integrating into the cellular genome, e.g., Raltegravir and Dolutegravir used for HIV; biologic drugs, e.g., interferons, which protect cells from being infected by viruses, antibody-based biologic drugs, e.g., casirivimab and imdevimab, used for COVID-19; steroids and anti-inflammatories, e.g., dexamethasone.

The term “viral infection” generally refers to the entry into and replication of a virus in a host cell. As would be appreciated by one of skill in the art, a “virus” is a small collection of genetic code, either DNA or RNA, surrounded by a protein coat and sometimes by a lipid membrane. A virus cannot replicate on its own and so must enter (i.e., infect) a host cell and use components of the host cell to make copies of itself.

Infections that are treatable or preventable with the compositions described in the present specification include those caused by, for example, RNA and DNA viruses. Examples include: Coronaviruses; Rhinoviruses; Respiratory Syncytial viruses; Influenza viruses; Ebola viruses (Zaire, Sudan, Bundibugyo, Tai Forest, and Reston); Marburg virus; Nipah virus; Zika virus; Human Immunodeficiency Virus (HIV); Lassa virus; Measles virus; Dengue virus; and Venezuelan Equine Encephalitis Virus.

As used here, the terms “treat,” “treatment,” and “treating” refer to the prevention, reduction or amelioration of the progression, severity and/or duration of a disorder, e.g., a viral infection, or the amelioration of one or more symptoms (preferably, one or more discernible symptoms) of the disorder, or the prevention of discernible infection, resulting from the administration of one or more therapies. In specific embodiments, the terms “treat,” “treatment,” and “treating” refer to the amelioration of at least one measurable physical parameter of a viral infection, such as reduction of viral load in a subject. In other embodiments, the terms “treat,” “treatment,” and “treating” refer to the inhibition of the progression of a viral infection, either physically by, e.g., stabilization of a discernible symptom, physiologically by, e.g., stabilization of a physical parameter, or both.

As described here, the term ‘subject’ or ‘patient’ refers to a warm-blooded animal such as a mammal that is afflicted with a particular disease, disorder or condition or is at risk of infection by a virus. It is explicitly understood that guinea pigs, dogs, cats, rats, mice, rabbits, horses, cattle, sheep, pigs, monkeys, chimpanzees, and humans are examples of animals within the scope of the term.

A “therapeutically effective amount” refers to an amount effective, at dosages and for periods of time necessary, to achieve the desired therapeutic result such as preventing, treating, or reducing the incidence of, a viral infection. An “amount effective” for treatment of a condition is an amount of an active agent or dosage form effective to achieve a determinable end-point related to preventing, treating or reducing the incidence of a viral infection. The “amount effective” is preferably safe - at least to the extent the benefits of treatment outweighs the detriments and/or the detriments are acceptable to one of ordinary skill and/or to an appropriate regulatory agency, such as the U.S. Food and Drug Administration. A therapeutically effective amount of a composition including the active agent, as described above, may vary according to factors such as the disease state, age, sex, and weight of the individual, and the ability of the active agent, as described above, to elicit a desired response in the individual. A therapeutically effective amount is also one in which any toxic or detrimental effects of compositions, including the active agent, as described above, are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

In general, a single therapeutically effective or prophylactically effective dose of the present composition will contain an amount of the active agent, as described above, in the range of about 0.01 to 30 mg/kg body weight, preferably about 0.05 to 20 mg/kg body weight, more preferably about 0.1 to 15 mg/kg body weight, and even more preferably about 0.5 to 10 mg/kg. For example, the amount of the active agent, as described above, in a single intravenous dose can be about 0.7 mg/kg, 1.1 mg/kg, 1.4 mg/kg, 1.8 mg/kg, 2.4 mg/kg, 2.8 mg/kg, 3.0 mg/kg, 3.2 mg/kg, 3.6 mg/kg, or more.

Administration of the active agent is typically via injection (e.g., infusion) of the suspension, and may be by any suitable injectable route, e.g., intravenous, intra-arterial, intradermal, intramuscular, intra-cardiac, intracranial, oral, nasal, intra-pulmonary, subcutaneous, or intraperitoneal. In appropriate situations, dry particles of the active agent, or a solution containing the active agent, could be aerosolized or nebulized and delivered via inhalation through the nose or mouth, or the solution could be delivered ocularly, nasally or orally as liquid, e.g. eye drops, nasal drops or spray. Also contemplated is oral delivery, e.g., as a mouthwash or gargle or sublingual formulation, or swallowed as a liquid, capsule, or tablet. Further details regarding routes of administration can be found, for example, in U.S. Pat. No. 7,834,147.

Administration can occur at least once a day for some number of consecutive days, e.g., for 3, 4, 5, 6, 7, 8, 9, or more consecutive days, or can be, e.g., every other day, or 3 times a week, or once every 7 ± 3 days, or once every 14 ± 3 days, or once every 28 ± 3 days. The timing of administrations can start with one of those schedules and after a suitable period of treatment change to another that is more or less frequent. The entire period of treatment may be completed in, e.g., eight or twelve or sixteen weeks, or up to six months, but may continue as long as the patient appears to be benefiting from the treatment and/or if the patient has been or is suspected to have been exposed to a virus. Where the treatment is for an ongoing infection, the treatment can end when it appears the patient’s active infection has ended, or can continue for 1 to 14 days longer. When the treatment is for prophylaxis, the treatment can begin at any point when prevention of infection is desired (e.g., when the patient’s risk of infection appears to be elevated, e.g., because of recent or expect exposure to the virus) and can end when the risk of infection appears to be lessened.

A given subject may receive a given dose level for one or more initial administrations and a different (lower or higher) level for further administrations. The delivery may be by any suitable route, e.g., intravenous, intra-arterial, intradermal, intramuscular, intracardiac, intracranial, subcutaneous, intraperitoneal, oral, intrapulmonary, nasal, transdermal, transmucosal, ocular, or topical. For certain of those routes (e.g., intrapulmonary, nasal, and oral), administration via aerosol spray or nebulizer may be useful.

### EXAMPLES

The invention is further described in the following examples, which do not limit the scope of what is claimed.

### Example 1: Antiviral Activity Against Influenza A H1N1

The activity of BXQ-350 against the Influenza A H1N1 subtype virus, a virus from the orthomyxoviridea family of viruses, as a representative of this class of viruses that can cause pandemics, was evaluated in the Influenza A/California/07/2009 (H1N1) pdm09 strain acquired from the American Type Culture Collection (ATCC). Influenza A viruses are an example of negative-sense, single-stranded, segmented RNA viruses.

The antiviral activity of BXQ-350 (expressed as EC₅₀) and the viability of cells treated with BXQ-350 (expressed as CC₅₀) were investigated in triplicate in MDCK cells using a range of concentrations between 60 and 0.25 microM. In this experiment, BXQ-350 was reconstituted in RPMI1640 supplemented with 1% non-essential amino acids (NEAA) and 1% penicillin-streptomycin. MDCK cells were maintained in the Minimum Essential Medium supplemented with 10% FBS, 1% L-glutamine, 1% NEAA and 1% penicillin-streptomycin. The OptiPRO SFM Medium supplemented with 1% L-glutamine, 1% NEAA and 1% penicillin-streptomycin was used as the assay medium. Cell viability was determined using a cell viability assay kit cell counting, and virally infected cells were determined using the cytopathic effect assay (CPE).

MDCK cells were seeded in 96 well plates, in 100 µl per well of assay medium, at a density of 15,000 cells per well, and cultured at 37° C. and 5% CO₂ overnight. The next day, the virus solution, in the OptiPRO assay medium, was added to each well (50 µl per well). BXQ-350, serially diluted with reconstitution medium to the desired concentration solutions, was then added to the wells (50 µl per well). The final volume of the cell culture was 200 µl per well and the final concentration of DMSO in the cell culture was 0.5%. The resulting cell culture experiments were incubated at 37° C. and 5% CO₂ for additional 5 days until virus infection in the virus control (cells infected with virus, without compound treatment) displayed significant CPE.

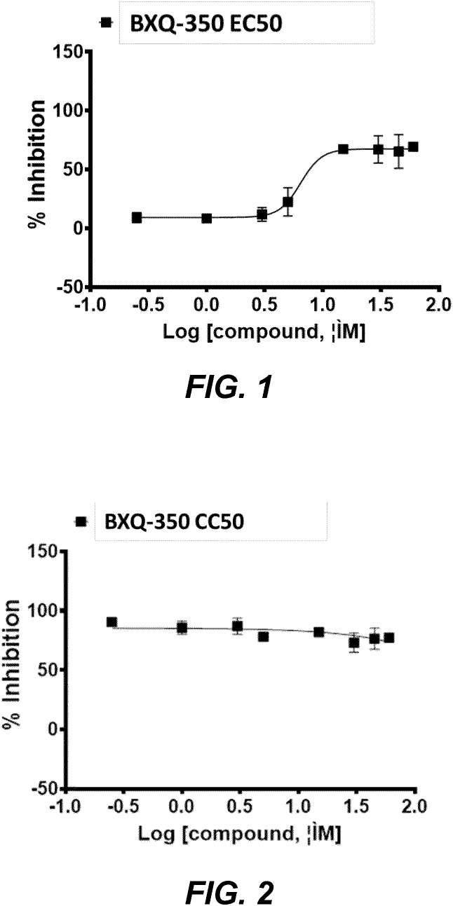

The antiviral activity of compounds was calculated based on the prevention of virus-induced CPE at each concentration, normalized by the virus control. The cytotoxicity of BXQ-350 was assessed in parallel, under the same conditions, but without virus infection. The EC₅₀ and CC₅₀ values were calculated and showed that BXQ-350 was highly potent against H1N1 and not toxic to normal cells: BXQ-350′s EC₅₀ was 4.94 microM as illustrated in FIG. 1; BXQ-350 was not toxic to the uninfected MDCK cells as shown in FIG. 2.

### Example 2: Antiviral Activity Against RSV

The activity of BXQ-350 against the Respiratory Syncytial Virus, a virus from the orthopneumovirus genus and pneumoviridae family of viruses that causes yearly epidemics, was evaluated in the RSA A long strain. RSV viruses are negative-sense, single-stranded RNA viruses.

The antiviral activity of BXQ-350 (EC₅₀) was investigated in triplicate in Hep-2 cells following the same methodology as described in Example 1 using the cytopathic effect to determine the antiviral activity of BXQ-350 against RSV. Cell cultures conditions were slightly different as Hep-2 cells were maintained in the Dulbecco’s modified Eagle Medium/F12 supplemented with 10% FBS and 1% penicillin-streptomycin. Hep-2 cells were seeded in 96 well plates, in 100 µl per well of assay medium, at a density of 6,000 cells per well and cultured at 37° C. and 5% CO₂ overnight. The next day, the virus solution, in the OptiPRO assay medium, was added to each well (50 µl per well; MOI=0.02). BXQ-350, serially diluted with reconstitution medium to the desired concentration solutions, was added to the wells after approximately one hour (50 µl per well). The final volume of the cell culture was 200 µl per well and the final concentration of DMSO in the cell culture was 0.5%. The resulting cell culture experiments were incubated at 37° C. and 5% CO₂ for additional 5 days until virus infection in the virus control (cells infected with virus, without compound treatment) displayed significant CPE.

The antiviral activity was calculated based on the protection of the virus-induced CPE at each concentration normalized by the virus control. BXQ-350′s EC₅₀ value against RSV A long was 5.41 microM, indicating that BXQ-350 was a potent inhibitor of RSV.

### Example 3: Antiviral Activity Against SARS-CoV

The activity of BXQ-350 against SARS-CoV, a virus from the coronavirus family, was investigated in the HCoV 229E strain acquired from the American Type Culture Collection (ATCC). The HCoV 229 E strain is an example of an enveloped, positive-sense, single-stranded virus that belongs to the class of alpha-coronaviruses.

The antiviral activity of BXQ-350 (EC₅₀) and viability of cells treated with BXQ-350 (CC₅₀) were investigated in triplicate in MRC-5 cells using a range of concentrations between 60 and 0.25 microM. In this experiment, BXQ-350 was reconstituted in RPMI1640 supplemented with 5% FBS, 1% NEAA and 1% penicillin-streptomycin. MRC-5 cells were maintained in the Minimum Essential Medium supplemented with 10% FBS, 1% L-glutamine, 1% NEAA and 1% penicillin-streptomycin. The fate of virally infected cells was determined using the cytopathic effect assay (CPE) to determine the inhibition properties of BXQ-350.

MRC-5 cells were seeded in 96 well plates, in 100 µl per well of assay medium, at a density of 15,000 cells per well and cultured at 37° C. and 5% CO₂ overnight. The next day, the virus solution, in the OptiPRO assay medium, was added to each well (50 µl per well). BXQ-350, serially diluted with reconstitution medium to the desired concentration solutions (90, 60, 45, 30, 15, 5, 1.5, and 0.25 microM), was then added to the wells (50 µl per well). The final volume of the cell culture was 200 µl per well and the final concentration of DMSO in the cell culture was 0.5%. The resulting cell culture experiments were incubated at 37° C. and 5% CO₂ for an additional 5 days until virus infection in the virus control (cells infected with virus, without compound treatment) displayed significant CPE.

The antiviral activity was calculated based on the protection of the virus-induced CPE at each concentration normalized by the virus control. The EC₅₀ values for BXQ-350 was calculated to be 4.47 microM, demonstrating that BXQ-350 was a potent inhibitor of SAR-CoV.

### Example 4: BXQ-350 Is Strongly Synergistic With Remdesevir Against SARS-CoV, HCoV 229E Strain

The effect of treatment with both BXQ-350 and Remdesivir against SARS-CoV (HCoV229E strain) was investigated in infected cells in vitro.

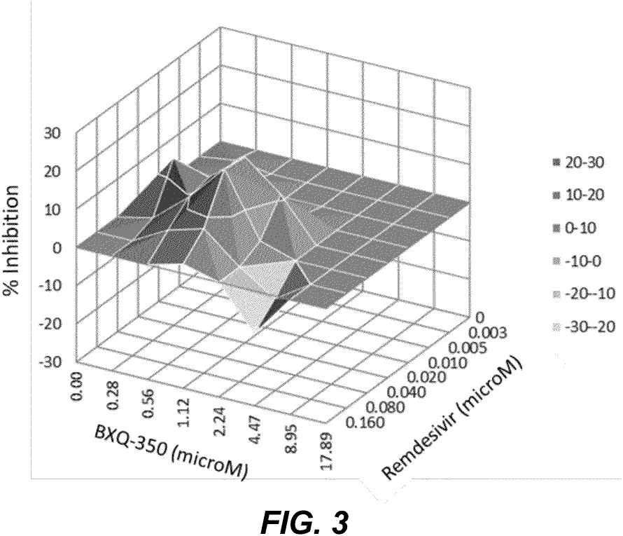

The experimental conditions were like those of Example 3. MRC5 cells were seeded in 96 well plates, in 100 µl per well of assay medium, at a density of 20,000 cells per well and cultured at 37° C. and 5% CO₂ overnight. The next day, cells were infected with 200 TCID₅₀ per well of virus. Solutions of BXQ-350 + Remdesivir) were tested based on a checkerboard pattern of 7 drug concentrations of each compound, including each compound alone or a total of 49 combinations (7*7); see Table 1 below for the concentrations of the compounds. The final volume of the cell culture was 200 µl per well and the final concentration of DMSO in the cell culture was 0.5%. The resulting cell culture experiments were incubated at 37° C. and 5% CO₂ for an additional 3 days until virus infection in the virus control (cells infected with virus, without compound treatment) displayed significant CPE. Plates were read using a Microplate Reader Synergy2, and the combinations indices were calculated using the MacSynergy II software. Positive combination indices are indicative of synergism, with a combination index value >+100 indicating strong synergism between these two compounds, without signs of toxicity as cell viability was between 96% and 105% across these combinations. A MacSynergy graph was also generated (FIG. 3); this graph allows a visualization of the synergism between these compounds.

TABLE 1

BXQ-350 and Remdesivir concentrations used

| | |
|---|---|
| BXQ-350 (microM) | Remdesivir (microM) |
| 17.89; 8.95; 4.47; 2.24; 1.12; 0.56; 0.28 and 0.0 | 0.16; 0.08; 0.04; 0.02; 0.01; 0.005; 0.003 and 0.0 |

TABLE 2

Combination Indices of BXQ-350 and Remdesivir against HCoV 229E

| Combination | Synergy Volume (95%) * | Antagonism Volume (95%) | Overall Combination Effect * |
|---|---|---|---|
| BXQ-350 + Remdesivir | 118.9 | -17.2 | Strong Synergism |

*an absolute combination index/synergy volume value >+100 indicates a strong synergism

### Example 5: Prophylactic Antiviral Activity Against SARS-CoV-2

The activity of BXQ-350 against SARS-CoV-2, a virus from the coronavirus family, was investigated in the SARS-CoV-2 KOR-KCDC03-2020 strain. SARS-CoV-2 is an example of an enveloped, positive-sense, single-stranded virus that belongs to the class of beta-coronaviruses.

The experimental conditions were similar to those used in the examples described above with a few changes that included: the cells used were Vero cells; Remdesivir was used as a positive control; an immunofluorescent assay, rather than the CPE assay, was used to detect virally infected cells; testing was performed in duplicate; and the test compounds, BXQ-350 and Remdesivir, were added to the cells about one hour prior to adding the viral inoculates to evaluate the prophylactic properties of BXQ-350, rather than its therapeutic properties as tested in the previous examples.

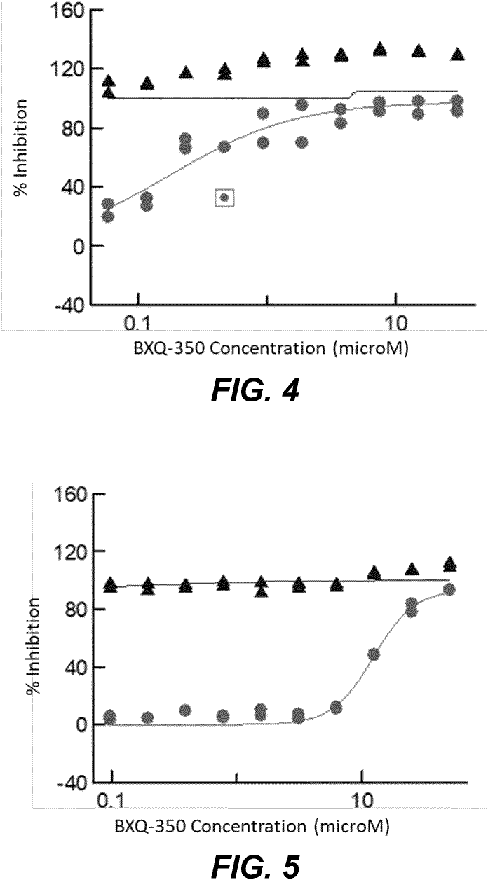

Vero E6 cells were seeded in 384-well plates, in 100 µl per well of assay medium, at a density of 12,000 cells per well. They were cultured at 37° C. and 5% CO₂ overnight. Solutions of BXQ-350 and Remdesivir were prepared by 2-fold serial dilutions starting from 50 microM down to 0.10 microM for Remdesivir and 30 microM down to 0.06 microM for BXQ-350. These drug solutions were added one day after the start of cell culture. One hour after the test compound solutions were added to the cells, the solution of SARS-CoV-2 was added to each well (MOI= 0.0125). The final volume of the cell culture was 200 µl per well and the final concentration of DMSO in the cell culture was 0.5%. The plate was incubated at 37° C. and 5% CO₂ for an additional 24 hours; at that time, a 4% paraformaldehyde solution was added to fix the cells. The plate was washed with PBS twice and treated with a 0.255% TritonX-100 solution for 20 minutes. The SARS-CoV-2 N antibody and Alexa Fluor® 488 goat IgG antibody in a 5% normal goat serum in PBS were added to the wells and the plate was incubated for one hour at 37° C. to detect virally infected cells. The images were analysed to quantify infected cells. Infection ratios and antiviral activity were normalized to positive (mock) and negative (0.5% DMSO) controls in each plate. As shown in Table 3 below and FIGS. 4 and 5, BXQ-350 is a potent inhibitor of SARS-CoV-2, significantly more potent that Remdesivir in these experimental conditions, and is not cytotoxic to Vero E6 cells.

TABLE 3

BXQ-350 and Remdesivir (Rem) EC₅₀ and CC₅₀ against SARS-CoV-2 virus in Vero cells

| Compound | EC₅₀ | CC₅₀ |
|---|---|---|
| BXQ-350 (microM) | 0.19 | >30.0 |
| Rem (microM) | 12.94 | >50.0 |

### Example 6: BXQ-350 Is Highly Synergistic With Remdesivir Against SAR-CoV-2

The synergism between BXQ-350 and Remdesivir against SARS-CoV-2 was also investigated. Using the same experimental conditions described in Example 5 above, solutions of BXQ-350 + Remdesivir were prepared by serial 2-fold dilutions starting from 50.0 microM for Remdesivir and 30 microM for BXQ-350 as shown in Table 4 below.

TABLE 4

BXQ-350 and Remdesivir concentrations used in combination
Concentrations (microM)

| | | | | | | | | | | |
|---|---|---|---|---|---|---|---|---|---|---|
| BXQ-350 | 30.0 | 15.0 | 7.50 | 3.75 | 1.88 | 0.94 | 0.47 | 0.23 | 0.12 | 0.06 |
| Remdesivir | 50.0 | 25.0 | 12.50 | 6.25 | 3.13 | 1.56 | 0.78 | 0.39 | 0.20 | 0.10 |

Solutions of (BXQ-350 + Remdesivir) were tested based on the 10 drug combinations listed above according to the Chou-Talalay method, a method used to determine the synergism between different drugs (see, e.g., Chou TC. Drug combination studies and their synergy quantification using the Chou-Talalay method. Cancer Res 2010;70:440-6). Briefly, this methodology compares the experimental values of inhibition by the combination to the calculated values of inhibition by each of the single compounds; an experimental inhibition result lower than the theoretical values indicates synergism between the test compounds while a value higher is indicative that the compounds are antagonistic; a combination index (CI) is calculated and synergism is represented by values lower than 1.0 (the lower the number, the more synergistic the compounds are).

Table 5 lists the EC₅₀ and CC₅₀ values calculated for the combination and Table 6 lists the combination indexes for the different combination tested. The results demonstrated that these two compounds are highly synergistic, as the CIs are well below 1.0.

TABLE 5

EC50 and CC50 results for BXQ-350 and Remdesivir (Rem) combination indicating a strong synergism between the two compounds.

| Compound | EC50 | CC50 |
|---|---|---|
| Rem + BXQ-350 | 0.33 | >50.0 |

TABLE 6

Combination Indexes (CI) for the BXQ-350+Remdesivir (Rem) combination.

| | | | | | | | | | | |
|---|---|---|---|---|---|---|---|---|---|---|
| Rem (mM) | 50.0 | 25.0 | 12.5 | 6.25 | 3.13 | 1.56 | 0.78 | 0.39 | 0.2 | 0.1 |
| BXQ-350 (mM) | 30.0 | 15.0 | 7.5 | 3.75 | 1.88 | 0.94 | 0.47 | 0.23 | 0.12 | 0.06 |
| CI | 0.140 | 0.070 | 0.035 | 0.017 | 0.218 | 0.396 | 0.380 | 0.831 | 1.51 | 2.25 |

When tested in a different cell line (Huh7 hepatic cells, a neoplastic cell line), BXQ-350 did not inhibit infection by a different strain of human coronavirus, OC43 HCoV. This was thought likely due to the fact that the BXQ-350 formulation necessitated assay modifications and/or Huh7 is a neoplastic cell line, which means it may have altered membrane characteristics compared to normal cells, and not to a lack of activity against that particular strain of virus.

### Example 7: Antiviral Activity Against Venezuelan Equine Encephalitis Virus

The activity of BXQ-350 against the Venezuelan Equine Encephalitis Virus, a virus from the Togaviridae family of viruses (genus Alphavirus, which includes both New World and Old World Alphaviruses), was evaluated in the VEEV TC-83 and VEEV TrD strains.

Using Vero cells, a 96 well plate was seeded with Vero cells to a confluency of 80%. BXQ-350 was added to the cells at concentrations ranging from 60 microM to 0.5 microM (in triplicate). Specifically, BXQ-350 was added to each well at the desired concentration for two hours prior to infection. Control wells with no drug added were also included. After two hours of incubation, the media containing the drug was removed and a fresh media was added with the virus to infect the cells using an MOI of 0.1 for 1 hour. The media containing the drug (media containing BXQ-350) was saved for the next step. After one hour, the media containing the virus was removed and the media of the previous step was added back (media containing BXQ-350) and cells were incubated for 24 hours. The next day, the supernatant from each well was recovered and saved for further processing by viral inhibition plaque assay.

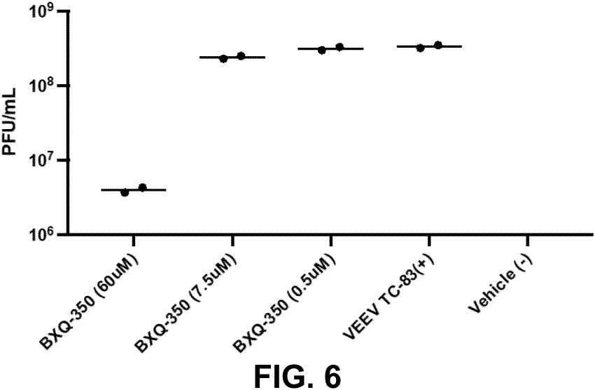

Antiviral activity of BX-350 was assessed based on the ability of BX-350 to inhibit viral replication. Viral replication was measured as PFU/ml (plaque forming units per mL of virus). FIG. 6 illustrates the antiviral properties of BXQ-350 as it depicts the inhibition of viral replication of the TC83 strain in Vero cells as a function of BXQ-350 Lower virus PFU/ml demonstrates inhibition of viral replication.

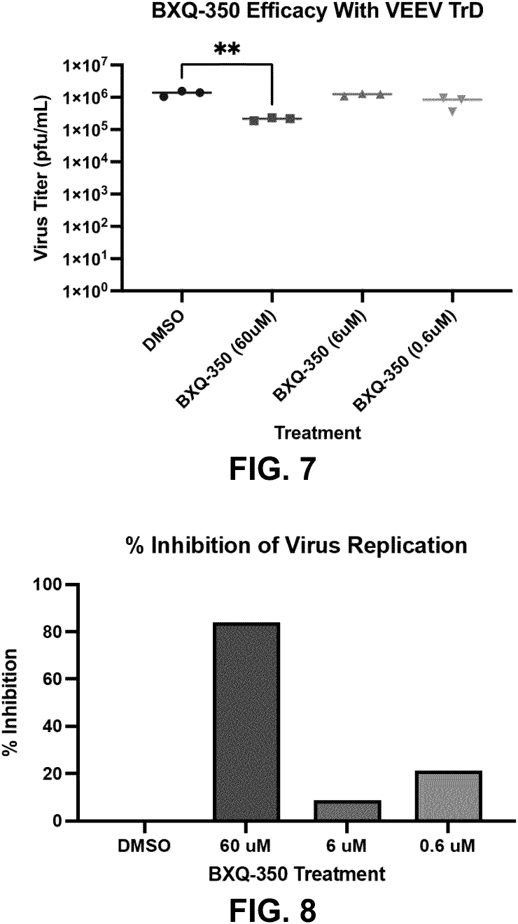

FIGS. 7 and 8, respectively, show the effect of BXQ-350 on the viral load (decrease in viral load or PFU/ml (plaque forming unit)) and associated percent inhibition against the virulent TrD strain.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. (canceled)

2. A method of reducing the incidence of a viral infection in a subject, the method comprising administering to the subject a prophylactically effective amount of an active agent comprising a saposin C polypeptide and a phospholipid.

3. (canceled)

4. (canceled)

5. The method of claim 2 further comprising an antiviral agent.

6. The method of claim 2, wherein the saposin C polypeptide comprises the amino acid sequence of SEQ ID NO: 1 with zero to four amino acid insertions, substitutions, or deletions.

7. The method of claim 2, wherein the saposin C polypeptide comprises the amino acid sequence of SEQ ID NO: 1.

8. The method of claim 2, wherein the saposin C polypeptide consists of the amino acid sequence of SEQ ID NO: 1.

9. The method of claim 2, wherein the phospholipid has a net negative charge at neutral pH.

10. The method of claim 2, wherein the phospholipid is a phosphoglyceride.

11. The method of claim 10, wherein the phosphoglyceride is a phosphatidate or phosphatidylserine.

12. The method of claim 11, wherein the phosphoglyceride is phosphatidylserine.

13. The method of claim 12, wherein the phosphatidylserine comprises one or more of dioleoyl phosphatidylserine (DOPS), dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, or diphytanoyl phosphatidylserine lipid, or a salt of any of the above.

14. The method of claim 13, wherein the phosphatidylserine is DOPS or a salt thereof.

15. The method of claim 2, wherein the molar ratio of the saposin C polypeptide to the phospholipid is in the range of 1:2 to 1:50.

16. The method of claim 15, wherein the ratio of the saposin C polypeptide to the phospholipid is 1:12.

17. The method of claim 2, wherein the viral infection is an infection by a virus selected from: SARS-CoV, MERS-CoV, SARS-CoV-2, Rhinovirus A, Rhinovirus B, Rhinovirus C, Poliovirus, Coxsackievirus, Echovirus, Enterovirus, Respiratory syncytial virus A, Respiratory syncytial virus B, Influenza A virus, Influenza B virus, Influenza C virus, Influenza D virus, Dhori virus, Ebolavirus, Marburgvirus, Nipah virus, a virus of the Flaviviridae family, and a virus of the Togaviridae family.

18. The method of claim 17, wherein the virus is Influenza A virus, Influenza B virus, Influenza C virus, or Influenza D virus.

19. The method of claim 17, wherein the virus is SARS-CoV-2.

20. The method of claim 17, wherein the virus is Respiratory syncytial virus A or Respiratory syncytial virus B.

21. The method of claim 2, wherein the active agent is administered intravenously in a dose of 2.3-3.2 mg/kg saposin C polypeptide.

22. The method of claim 2, wherein a dose of the active agent is administered to the subject each day for at least three consecutive days.

23. The method of claim 2, wherein the subject is a human.

24. The method of claim 5, wherein antiviral agent is remdesivir.


SEQUENCE LISTING (1 sequence)

SEQ ID NO: 1 - protein, 80 aa, Homo sapiens
SDVYCEVCEFLVKEVTKLIDNNKTEKEILDAFDKMCSKLPKSLSEECQEVVDTYGSSILSILLEEVSPELVCSMLHLCSG